(19) United States
(12) Patent Application Publication
(10) Pub. No.: US 2025/0275567 A1
(43) Pub. Date: Sep. 4, 2025

(54) NUTRITIONAL SUPPORT SYSTEM TO ACTIVATE ALTERED METABOLIC PATHWAYS

(71) Applicant: Fernando LEAL MARTINEZ, Mexico (MX)

(72) Inventor: Fernando LEAL MARTINEZ, Mexico (MX)

(21) Appl. No.: 18/278,846

(22) PCT Filed: Feb. 22, 2022
(86) PCT No.: PCT/MX2022/050009

§ 371 Date: Aug. 25, 2023

(30) Foreign Application Priority Data
Feb. 26, 2021 (MX) MX/A/2021/002357

(51) Int. Cl. A23L 33/00; A23L 2/38; A23L 2/52; A23L 33/10

(52) U.S. Cl. CPC A23L 33/40; A23L 2/38; A23L 2/52; A23L 33/10

(57) ABSTRACT

A comprehensive nutritional support system comprising a nutraceutical composition and/or dietary composition and/or dietary supplement and/or food supplement and/or food supplement and/or therapeutic composition for oral administration of nutrients at therapeutic doses useful for the prevention and/or treatment and/or recovery of a mammal suffering from a chronic and/or degenerative disease state and/or viral infection that is unable to attain the nutritional requirements of an average normal diet. This novel system of integral nutritional support is characterized by the highly novel combination to potentiate the treatment of at least one disease suffered by the mammal patient.

### FIELD OF THE INVENTION

The present invention relates to the field of health, and of biotechnology, more particularly to the field of nutrition, as it is intended to provide a comprehensive nutritional support system comprising a nutraceutical composition and/or nutraceutical formulation and/or dietary composition and/or dietary supplement and/or food supplement and/or food supplement and/or orally administered therapeutic composition.

The present novel invention is useful for the prevention and/or treatment and/or recovery based on a diet and/or nutritional mixture of the type of nutraceutical composition and/or dietary composition and/or dietary supplement and/or food supplement and/or food supplement and/or acceptable therapeutic composition that is employed when a mammal presenting a chronic and/or degenerative disease state is unable to attain the nutritional requirements of an average normal diet, such as may be: rejection or inability of the mammal to food alone and/or commonly prepared without a specific diet system, need for addition of at least one fiber, need for addition of at least one mineral and at least one vitamin, need for addition of at least one prebiotic and/or probiotic, need for addition of at least one cyanobacterium, need for addition of at least one amino acid, need for addition of at least one animal and/or vegetable protein, need for addition of at least one plant, need for addition of at least one root, need for addition of at least one polyunsaturated fatty acid and/or at least one vegetable oil, need for substitution of whole and/or lactose-free and/or light milk from cow, goat, sheep, donkey or other animals by milks derived from fruits and/or seeds and/or plants and/or legumes.

This novel system of integral nutritional support is characterized by the highly novel combination of nutrients at therapeutic doses to cover nutritional deficiencies and activate metabolic pathways that are altered, where such metabolic pathway activation is conducted through the activation of epigenetics to potentiate the treatment of at least one disease suffered by the mammalian patient. The nutraceutical composition and/or dietary composition and/or dietary supplement and/or food supplement and/or food supplement and/or therapeutic composition provides the symbiosis of all its ingredients at therapeutic doses through the administration of the nutritional support system for the purpose of prevention and/or treatment and/or recovery effects of diseases in mammals. This novel invention also offers the following surprising benefits: it favors neurological, motor, and visual development in the short and long term, including IQ, mental development index and problem solving. It stimulates immunity, regulates inflammation, favors the recovery of patients with pneumonia and improves the prognosis against viral infections such as SARS-CoV-2.

### BACKGROUND

Nutrition is the science that studies how the body uses energy and substrates from food to maintain itself and grow, by analyzing the processes by which it ingests, digests, absorbs, transports, utilizes and extracts nutrients essential for life, genetics, cell replication and repair, and their interaction with health and disease.

Diet is a fundamental determinant of the state of health of individuals. Essential nutrients include minerals, vitamins, lipids, amino acids, proteins, healthy fatty acids, whose deficient intake gives rise to the characteristic clinical pictures of deficiency diseases, such as beri-beri due to thiamine deficiency (vitamin B1), rickets due to vitamin D deficiency, iron deficiency anemia, genetic and neurological alterations due to folate and cobalamin deficiency, among other diseases. As nutritional supplements, the addition of certain ingredients such as probiotics and prebiotics is required to improve the gastrointestinal system.

Pregnancy is a vitally important and complex period, where the future being, in addition to increasing cell mass, develops and matures morphologically to progressively acquire functional capacities. A multitude of factors come together in pregnancy that imply that the life of a future being may fail or develop with total or partial success.

Nutrition in pregnancy, in addition to providing nutrients and other substances that ensure the replacement of structures and growth, is a process where the energetic, structural and metabolic control and regulation functions are due to the interaction of nutrients on our genes, which will enable gene expression and the formation of biochemical “entities” that make possible the best functionality of the nutrients.

Breast milk is the ideal food during the first months of life for the development of the child due to its nutritional, immunological, psycho-affective and economic characteristics, among others.

In nutrition, the interest of the diet has been fixed on the current epidemic of diseases such as cardiovascular diseases, obesity, diabetes, cancer, dementia, gastrointestinal, bone, congenital malformations (during the first weeks of birth) and congenital diseases (developed before birth). Worldwide, gastrointestinal infections are one of the most important causes of morbidity and mortality among infants and children, since the intestine regulates inflammation, immunity and metabolism. In Mexico, gastrointestinal diseases are one of the main public health problems. Gastrointestinal disorders are becoming more common every day, affecting mainly children, and both their incidence and prevalence depend on the socioeconomic level of the population.

depend on the socioeconomic level of the patients. They can affect one or more parts of the same gastro intestine, causing some disorder among which include: diarrhea, regurgitation, rumination syndrome, cyclic vomiting syndrome, colic, infantile dyschezia, aerophagia, gastroenteritis or gastric atrophy, constipation, abdominal pain, ulcerative colitis according to the region of the intestine; these can cause growth failure, hematemesis, anemia, refusal to feed, malnutrition, excessive crying, among others (Wilson D. 2010).

For the treatment of said gastrointestinal diseases, nutrients such as zinc, vitamin C and probiotics are used including Bacteroides sp, Bifidobacterium longum, Bifidobacterium spp., Bifidobacterium infantis, Bifidobacterium breve, Lactobacillus spp., Lactobacillus salivarius, Lactobacillus bulgaricus, Saccharomyces boulardii, Streptococcus thermophilus, Lactobacillus acidophilus, Lactobacillus Casei, Lactobacillus brevis y Lactobacillus Plantarum and/or prebiotics such as non-starch polysaccharides, inulin, lactulose, fructo-oligosaccharides, galacto-oligosaccharides, resistant starch, breast milk oligosaccharides, among others; cereal and/or nut derivatives such as amaranth, rye, almonds, hazelnuts, peanuts, coconut, among others.

The combination of probiotics is useful to maintain intestinal health, restore the bacterial flora, reduce cholesterol in the blood circulation and stimulate the body's immune system. The use of prebiotics allows the selective stimulation of the growth and/or activity of a type or number of bacteria, producing beneficial effects on the host.

The use of the combination of probiotics and prebiotics produces symbiosis and/or synergism on protective effects, prevention of gastrointestinal infections, among others.

Proteins are fundamental to the cells of the human body. The basic structure of protein is a chain of amino acids, which are found in animal sources such as meat, milk, fish and eggs. They are also found in plant sources such as brewer's yeast, microalgae, amaranth, soybeans, beans, legumes, nut butters and some grains such as wheat germ and quinoa.

Amino acids are classified into three groups: Essential, Non-essential and Conditional.

Essential amino acids cannot be produced by the body and must be provided by food, so their intake throughout the day is very important; non-essential amino acids are produced by the body from essential amino acids or in the normal breakdown of proteins and conditional amino acids are necessary in times of illness and indispensable for genetics, epigenetics and cellular and immune replication.

Lipids are an essential part of a healthy diet, structural components of utmost importance for vital organs, particularly for mental and visual development.

Long-chain polyunsaturated fatty acids support development, including docosahexaenoic acid (DHA) and arachidonic acid (ARA), which are of significant importance for proper mental development at the level of the central nervous system and for visual development at the level of the retina.

Short chain fatty acids are essential for the communication established between the intestine and the brain and between the lung and the brain; they stimulate immunity and regulate the inflammatory response, mediated by the cytokine storm. DHA is an omega-3 fatty acid, which is a functional element of all membranes in the gray matter of the brain, eyes and heart tissues. ARA is an omega-6 fatty acid, which promotes organ and tissue growth, is a precursor of a group of hormone-like substances such as eicosonoids and participates in the development of the immune system.

Vegetable oils are composed of lipids, the most important of which are fatty acids and some vitamins. Among the main benefits are: reduction of cholesterol, optimal functioning of the cardiovascular system, maintenance of the reproductive and nervous systems, improvement of hair and skin.

Vegetable oil is a substance that has been obtained from fruits, seeds, cereals, trees, among others, composed of a high content of monounsaturated or polyunsaturated fatty acids of several types, conferred by the properties of the fruit, seeds, cereals, trees, among others. Among the main oils used are amaranth, flaxseed, chia, walnut, sunflower, linseed, rapeseed, castor, soybean, peanuts, walnuts, almonds, cotton, peanuts, among others.

Vegetable oils provide beneficial effects such as cardiovascular system optimization, micronutrient absorption, cholesterol reduction, prevention and treatment of cardiovascular diseases.

Bran is the refined product of cereal grains, corresponding to the outer layers, which are rich in fiber, vitamins, minerals, enzymes and essential fatty acids. Examples are cereals such as wheat, rice, barley, oats, rye, corn, millet, sorghum and buckwheat, legumes (pulses), starchy roots (such as rice, cassava).

Oat and wheat bran have beneficial properties, including cholesterol control, sugar control and toxin absorption. The following table describes the nutrient content of each bran:

| | Raw wheat bran | | Raw oat bran | | Raw rice bran | | Raw corn bran | |
|---|---|---|---|---|---|---|---|---|
| Water | 9.89 | g | 6.55 | g | 6.13 | g | 4.71 | g |
| Calories | 216 | kcal | 246 | kcal | 316 | kcal | 244 | kcal |
| Fat | 4.25 | g | 7.03 | g | 20.85 | g | 0.92 | g |
| Proteins | 15.55 | g | 17.30 | g | 13.35 | g | 8.36 | g |
| Carbohydrates | 64.51 | g | 66.22 | g | 46.69 | g | 85.64 | g |
| Fibers | 42.8 | g | 15.4 | g | 21 | g | 85.5 | g |
| Potassium | 1182 | mg | 566 | mg | 1485 | mg | 44 | mg |
| Phosphorus | 1013 | mg | 734 | mg | 1677 | mg | 72 | mg |
| Iron | 10.57 | g | 5.41 | g | 18.54 | mg | 2.79 | mg |
| Sodium | 2 | mg | 4 | mg | 5 | mg | 7 | mg |
| Magnesium | 611 | mg | 235 | mg | 781 | mg | 64 | mg |
| Calcium | 73 | mg | 58 | mg | 57 | mg | 42 | mg |
| Copper | 0.998 | mg | 0.403 | mg | 0.728 | mg | 0.248 | mg |
| Zinc | 7.27 | mg | 3.11 | mg | 6.04 | mg | 1.56 | mg |
| Manganesium | 11.500 | mg | 5.630 | mg | 14.210 | mg | 0.140 | mg |
| Vitamin A | 0 | mg | 0 | mg | 0 | mg | 71 | UI |
| Thiamine | 0.523 | mg | 1.170 | mg | 2.753 | mg | 0.010 | mg |
| Riboflavin | 0.577 | mg | 0.220 | mg | 0.284 | mg | 0.100 | mg |
| Niacin | 13.578 | mg | 0.934 | mg | 33.995 | mg | 2.735 | mg |
| Pyridoxine | 1.303 | mg | 0.166 | mg | 4.070 | mg | 0.152 | mg |
| Vitamin E | 2.320 | mg | 1.720 | mg | 6.050 | mg | 2.320 | mg |
| Folic acid | 79 | mcg | 52 | mcg | 63 | mcg | 4 | mcg |

In the prior art, compositions can be found comprising: a nutraceutical product with an encapsulated primer containing Lactobacillus acidophilus, Bifidobacterium bifidum, a second encapsulation containing DHA and ARA among others, corresponding to patent U.S. Pat. No. 8,221,809; A composition for food use comprising: a) A mixture of freeze-dried live bacteria comprising at least two species of bacteria selected from Bifidobacterium breve, Bifidobacterium infantis, Bifidobacterium longum and Bifidobacterium bifidum and at least two species of bacteria selected from Lactobacillus acidophilus, Streptococcus thermophilus, Lactobacillus case, Lactobacillus plantarum and Streptococcus faecium; b.) One or more oligosaccharides; said component (a) being present in an amount of 4 to 20 parts by weight and said component (b) being present in an amount of 5 to 22 parts by weight, further comprising a component consisting of sugar or an artificial sweetener, a component consisting of a dispersant in the form of powder, particles or fibers of natural products corresponding to patent MX202987; A composition containing bacteria and fiber to promote gastrointestinal health comprising: a) An effective amount of beneficial human intestinal microorganisms; and b) An effective amount of dietary fiber, wherein said dietary fiber is a member selected from the group consisting of pentosans, beta-glucans, pectins and pectic polysaccharides, mannans, arabins and galactans, fructooligosaccharides and mixtures thereof, for treating gastrointestinal diseases corresponding to patent U.S. Pat. No. 6,241,983; pharmaceutical compositions containing Lactobacillus acidophilus, Bifidobacterium bifidus, Streptococcus lactis, polyunsaturated fatty acids, vitamins, growth factors, among other compounds, administered with milk for the treatment of gastrointestinal diseases and cancer disorders corresponding to patent application WO/2003/022255; Use of a probiotic or probiotic mixture in the preparation of a nutritional composition or drug to act on the colon in a pre- or post-surgical setting, for the prevention and management of non-infectious diarrhea, of post-surgical abdominal-pelvic infections due to pelvic fluid collection secondary to anastomotic leakage or bacterial translocation, for the prevention or relief of gastrointestinal symptoms secondary to global changes in the intestinal microbial ecology and metabolic activity of the microbiota, preferably infectious or toxigenic diarrhea, prevention or treatment of gastrointestinal infections, preferably nosocomial gastrointestinal infections, to modulate the inflammatory response, in particular during the healing process, to modulate the immune system and/or to stimulate the production of mucosal secretory antibodies, wherein the selected probiotic consists of bifidobacterium, lactobacillus, streptococcus and saccharomyces or mixtures thereof, further comprising non-viable probiotic bacteria and/or probiotic-derived material, further comprising prebiotics, a source of carbohydrates, a source of lipids and a source of protein and micronutrients, selected from at least vitamin E and vitamin C, corresponding to patent application MX/a/2009/012255; A dietary supplement or pharmaceutical composition, comprising lyophilized Saccharomyces boulardii as the active ingredient, optionally in association with a pharmaceutically acceptable vehicle, wherein the composition is in a closed bottle having a first airtight compartment comprising lyophilized S. boulardii powder and a second compartment comprising a solution, wherein the first and second compartments may be in airtight communication with each other to produce a suspension of S. boulardii suspension to be administered to an individual upon opening, the bottle, wherein the first compartment also comprises lactose, magnesium stearate, at least one mineral and/or at least one vitamin, for use in maintaining the balance of the intestinal flora, for maintaining the proper functioning of the intestines, to maintain normal bowel function and/or to promote intestinal health, in the prevention or treatment of microbial imbalance of the digestive tract, diarrhea and/or a bacterial, fungal or protozoan infection, corresponding to patent application MX/a/2016/016387; Use of a gelatinous mixture of probiotics and prebiotics with synergistic symbiotic action where the components of the gelatinous mixture are: water, cane sugar, glucose in liquid state, protein element, xanthan gum, blue agave fructans (agave tequila Weber), vitamins, citrus seed extract, citric acid, malic acid, bifidobacterial (bifidus), lactobacilli, colorant, essence; for the manufacture of a coadjuvant food in the treatment of patients with chronic kidney disease, where the food after being adapted for consumption over 6 months reduces by 71% the levels of urea in the blood, which after being adapted for consumption over 6 months delays the start of dialysis therapy corresponding to patent MX367504; A nutritional composition, comprising: A lipid or fat source, a protein source, and a probiotic stabilized in a protective matrix, comprising: i) A hydrolyzed protein comprising hydrolyzed casein, ii. A first carbohydrate selected from sucrose, maltose, lactose, trehalose, 5 maltotriose, maltodextrin having dextrose equivalent of about 2 through 6, and combinations, iii A second carbohydrate selected from inulin, polydextrose, galactooligosaccharide, fructooligosaccharide, starch, maltodextrin, having a dextrose equivalent greater than 8, and combinations, wherein the probiotic comprises viable microbial cells selected from Lactobacillus rhamnosus, the matrix comprises: Sodium alginate, pectin, and a lipid selected from: lecithin, monoglycerides, diglycerides, and combinations, said nutritional composition is a powdered infant formula corresponding to patent application MX/a/2016/007007.

All the compositions described in the prior art are characterized in that their use is for the specific treatment of some disease or disorder of the gastrointestinal, respiratory, immune, cancerous system, among others.

No comprehensive nutritional support system based on a nutraceutical composition and/or dietary composition and/or dietary supplement and/or food supplement and/or food supplement and/or orally administered therapeutic composition containing the combination of the following groups of functional ingredients at therapeutic doses, selected from: high levels of vegetables, fruits, cereals, tubers and proteins. Additionally, it is supplemented with amino acids, vitamins, minerals and/or trace elements, cyanobacteria, vegetable protein, n-3 PUFAs, probiotics and/or prebiotics, seeds, cereals, trees and/or tree bark and/or tree stems and/or tree leaves and/or roots, plants and/or plant stems and/or plant leaves and/or roots, plant milk, polyunsaturated fatty acids including docosahexaenoic acid (DHA) and arachidonic acid (ARA).

Said integral nutritional support system is useful for the prevention and/or treatment and/or recovery of diseases in mammals selected from: gastrointestinal diseases and/or cardiovascular diseases and/or immune system deficiency and/or neuronal diseases, either congenital or non-congenital.

### OBJECT OF THE INVENTION

The objective of the invention is to provide a comprehensive nutritional support system based on a nutraceutical composition and/or dietary composition and/or food supplement and/or food complement and/or therapeutic composition for oral administration, characterized in that it comprises the combination of the following groups of functional ingredients at therapeutic doses, selected from: high levels of vegetables, fruits, cereals, tubers and proteins of animal and/or vegetable origin. Additionally, it is supplemented with amino acids, vitamins, minerals and/or trace elements, cyanobacteria, vegetable protein, n-3 PUFAs, probiotics and/or prebiotics, seeds, cereals, trees and/or tree bark and/or tree stems and/or tree leaves and/or tree roots, plants and/or plant stems and/or plant leaves and/or plant roots, vegetable and/or animal milk, polyunsaturated fatty acids including docosahexaenoic acid (DHA) and arachidonic acid (ARA), and/or nutritionally acceptable excipients. Said integral nutritional support system is useful for the prevention and/or treatment and/or recovery of diseases in mammals selected from: gastrointestinal diseases and/or cardiovascular diseases and/or immune system deficiency and/or neurological diseases, either congenital or non-congenital. This nutraceutical composition acceptable, stable and easily digestible for mammals, is characterized by the amazing benefits it offers, the prevention and/or treatment and/or recovery of gastrointestinal diseases such as diarrhea, ulcerative colitis, constipation, gastroenteritis or gastric atrophy; restoration of the bacterial flora such as good bacteria of the large and small intestine region; prevention and/or treatment of cardiovascular diseases and/or cholesterol control; mental and visual development; selected motor development of cerebral palsy; stimulation and strengthening of the immune system against bacteria, viruses, fungi and other pathogenic microorganisms.

### BRIEF DESCRIPTION OF THE DRAWINGS

FIG. 1. Consort, transparent assay report.

FIG. 2. GMFD (standing) evaluations of FG, CG, and IG

FIG. 3. GMFE (walking) evaluations of FG, CG and IG.

FIG. 4. Percentage of evolution (GMFD, standing) of FG, CG and IG.

FIG. 5. Percentages of evolution (GMFE, walking) of FG, CG and IG.

FIG. 6. Overall survival by COVID-19 of the GC and GI.

FIG. 7. Overall survival by COVID-19 of the GC and G

FIG. 8. Survival in patients intubated by COVID-19 of the GC and GI.

### DETAILED DESCRIPTION OF THE INVENTION

The present invention refers to a novel system of integral nutritional support based on a nutraceutical composition and/or dietary composition and/or dietary supplement and/or food supplement and/or food complement and/or therapeutic composition at therapeutic doses in the form of powder and/or lyophilized powder and/or semi-solid and/or liquid and/or in natural state designed to be administered orally, indicated for mammals, more specifically human beings in the stages of: infants (babies), children, adolescents, adults and elderly adults. This novel invention also offers the following benefits: it favors neurological, motor and visual development in the short and long term, including IQ, mental development index and problem-solving ability. It stimulates immunity, regulates inflammation, favors the recovery of patients with pneumonia and improves the prognosis against viral infections such as Sars-CoV-2. It is useful for the prevention and/or treatment and/or recovery of diseases in humans, selected from: gastrointestinal diseases and/or cardiovascular diseases and/or immune system deficiency and/or neurological diseases, either congenital or non-congenital.

This nutraceutical composition acceptable, stable and easily digestible for humans, is characterized by the benefits it offers, prevention and/or treatment and/or recovery of gastrointestinal diseases such as diarrhea, ulcerative colitis, constipation, gastroenteritis or gastric atrophy; restoration of the bacterial flora such as good bacteria of the large and small intestine region; prevention and/or treatment of cardiovascular diseases and/or cholesterol control; mental and visual development; selected motor development of cerebral palsy; stimulation and strengthening of the immune system against diseases caused by bacteria, viruses, fungi and other pathogenic microorganisms. This comprehensive nutritional support system is focused on, but not limited to, gastrointestinal diseases caused by parasites, mental motor diseases such as cerebral palsy and immune diseases caused by selected viruses from the group consisting of Arenaviridae, Coronaviridae, Filoviridae, Flaviviridae and Paramyxoviridae, more specifically by selected Coronaviridae group viruses of SARS (SARS-CoV-1 and SARS-CoV-2), MERS, 229E, NL63, OC43, and HKU1. Coronaviridae group viruses are a group of RNA (ribonucleic acid) viruses that cause respiratory and digestive diseases, producing mild to severe symptoms such as acute respiratory distress syndrome and death. The SARS-CoV-2 virus type causes the disease called coronavirus disease 2019 (COVID-19), first manifested in Wuhan, China, in December 2019.

Said integral nutritional support system based on a nutraceutical composition and/or dietary composition and/or food supplement and/or food complement and/or therapeutic composition for oral administration, is characterized in that it comprises the combination of several groups of ingredients at therapeutic doses under a rigorous balance of nutrient supply necessary to carry out the biochemical and epigenetic reactions, favoring the patient's recovery.

Said group of functional ingredients at therapeutic doses, are selected from: at least one vegetable and/or vegetable, at least one fruit, at least one cereal, at least one tuber, at least one protein of animal and/or vegetable origin, at least one lipid of animal and/or vegetable origin; and combinations thereof. Additionally, it is supplemented with at least one amino acid of natural and/or synthetic origin, at least one carbohydrate of natural and/or synthetic origin, at least one vitamin of natural and/or synthetic origin, at least one mineral and/or macromineral and/or trace element and/or micronutrient, at least one cyanobacterium and/or algae and/or yeast, at least one protein of animal and/or vegetable origin, at least one n-3 PUFAs, at least one probiotic and/or at least one prebiotic, at least one seed, at least one cereal, at least one tree and/or tree bark and/or tree stem and/or tree leaves and/or tree roots, at least one plant and/or plant stem and/or plant leaves and/or plant roots, at least one vegetable and/or animal milk, at least one animal and/or vegetable lipid, at least one polyunsaturated fatty acid, at least one cereal and/or cereal derivative and/or at least one dried and/or dehydrated fruit and/or at least one drug and/or excipients and/or nutritionally acceptable excipients and/or nutritionally acceptable vehicles.

In accordance with the above described, at least one vegetable and/or greens is selected from: Swiss chard, garlic, artichoke, basil, artichoke, artichoke, celery, celeriac, celeriac, ascalonia, pea, sweet potato, eggplant, beet, broccoli, sweet potato, borage, zucchini, squash, pumpkin, cardoon, parsnip, onion, mushroom, chayote, corn, green beans, white cabbage, Chinese cabbage, savoy cabbage, red cabbage, kohlrabi, kohlrabi, Brussels sprouts, pumpkin, cauliflower, shallots, endive, shallots, endive, curly endive, asparagus, spinach, beans, peas, lima beans, mushrooms, lamb's lettuce, tomato, ginger, green beans, red cabbage, corn, turnip, potato, parsley, cucumber, paprika, bell pepper, leek, radish, beet, mushroom, tomato, carrot, nopal, cucumber, bean sprouts, bean, lettuce, fennel, radish, pumpkin or combinations thereof.

At least one fruit is selected from: apple, orange, banana, coconut, tangerine, avocado, pear, melon, watermelon, lemon, guava, guava, tejocote, peach, grape, plum, blueberry, strawberry, quince, papaya, pomegranate, mango, cane, pineapple, jicama, raisins, prunes, blackberries, raspberries, figs or combinations thereof.

At least one cereal is selected from amaranth, oats, brown rice, barley, millet, corn, kasha or buckwheat, wheat, spelt, bulgur, teff, quinoa, rye, sorghum, triticale, bran or combinations thereof. At least one leaf is selected from watercress, chard, spinach, lettuce, endive, chicory, kale, arugula, arugula, cilantro, parsley, quelites, quintoniles, lettuce or combinations thereof.

At least one root or tuber is selected from broccoli, turmeric, ginger, radish, potato, carrot, turnip, sweet potato, beet, onion, garlic, celery, parsnips, leek, licorice, ginseng, valerian, cassava or mixtures thereof.

At least one stalk is selected from chard, celery, borage, bamboo shoots, fern shoots, cinnamon, thistle, kohlrabi, asparagus, scallions, cilantro, fennel, hearts of palm, parsley, leek, rhubarb, or mixtures thereof. At least one animal protein is selected from tuna, lamb, kid, goat, pork, rabbit, turkey, chicken, veal, tilapia, beef, anchovy, salmon, mussels, sardines, sea bass, swordfish, cuttlefish, shrimp, cod, shrimp, egg, cheese, natural yogurt, milk, kefir, skyr or combinations thereof.

At least one protein of plant origin is selected from mushrooms, chickpeas, peanuts, pistachios, lentils, red lentils, brown lentils, beans, soybeans, quinoa, peas, spirulina algae, sesame seed, seitan, hemp seeds, pine nuts, pumpkin seeds, almonds, red beans, black beans, sunflower seeds, chia seeds, flaxseed, tempeh, amaranth, walnuts, oats, hazelnuts, buckwheat, brewer's yeast, rice or combinations thereof.

At least one lipid and/or fat and/or oil and/or fatty acid of animal origin is selected from lard, bacon, bacon, salami, beef, sheep, goat, fish, cod, shark, salmon, anchovy, sardine, whale and/or their milk products, cream, cheese, yogurt, butter or combinations thereof.

At least one lipid and/or fat and/or oil and/or fatty acid of vegetable origin is selected from olive, avocado, sesame seed, grape seed, coconut, shea, marigold, cinnamon, rose, palm, soybean, rice, corn, flaxseed, safflower, hemp, canola, almond, walnut, hazelnut, pumpkin seed, castor, chia, cottonseed or combinations thereof.

At least one amino acid of natural and/or synthetic origin is selected from histidine, isoleucine, leucine, lysine, methionine, phenylalanine, threonine, tryptophan, valine, alanine, arginine, asparagine, aspartic acid, cysteine, glutamic acid, glutamine, glycine, proline, serine, tyrosine, ornithine, proline, serine or combinations thereof.

At least one carbohydrate of natural and/or synthetic origin is selected from ribose, deoxyribose, sucrose, lactose, maltose, cellobiose, glycoproteins, starch, amylose, glycogen, cellulose, chitin, corn, potato, beans, inulin or combinations thereof.

At least one vitamin of natural and/or synthetic origin is selected from A, B (thiamin, riboflavin, niacin, pantothenic acid, biotin, vitamin B-6, vitamin B-12 and folate or folic acid), C, D, E, K, beta-carotene, or combinations thereof.

At least one mineral of natural and/or synthetic origin are selected from calcium, phosphorus, magnesium, sodium, potassium, chlorine, sulfur, iron, magnesium, copper, iodine, zinc, cobalt, fluorine, boron, chromium, manganese, selenium, vanadium, lutein, silicon or combinations thereof.

At least one cyanobacterium and/or algae and/or yeast is selected from Arthrospira sp., Crypthecodinium cohnii, Rhodotorula glutinis, Spirulina, Nostoc, Chlorella, brewer's yeast or mixtures thereof. At least one PUFA is selected from linolenic acid, omega 3 and omega 6, omega 9 or combinations thereof.

At least one prebiotic is selected from fructooligosaccharides (FOS) or fructans, maltodextrin, resistant starch, lactulose and galactooligosaccharides (GOS) from breast milk. Other main sources of prebiotics are selected from garlic, onions, artichokes, bananas, honey, wheat, oats, soybeans, legumes, asparagus, chicory, inulin, leeks or combinations thereof.

At least one probiotic is selected from Saccharomyces boulardii, Lactobacillus casei, Lactobacillus rhamnosus, L. delbrueckii subsp. Bulgaricus, Lactobacillus, Bifidobacterium, Bifidobacterium breve, Bifidobacterium infantis, Bifidobacterium longum and Bifidobacterium bifidum, Lactobacillus acidophilus, Streptococcus thermophilus, Lactobacillus case, Lactobacillus plantarum and Streptococcus faecium or mixtures thereof.

At least one seed is selected from chia, almond, sunflower, pumpkin, sesame, linseed, poppy, fennel, moringa, peanut, corn, walnut, chestnut, chestnut, acorn, hazelnut, quinoa, grape, ojoche, cumin, hemp, apricot, pomegranate, hemp, pine nut, wild rice, lotus or combinations thereof.

At least one milk of vegetable origin is selected from coconut, walnut, macadamia nut, almond, soybean, pea, amaranth, rice, oat, quinoa, canary seed or combinations thereof.

At least one milk of animal origin is selected from cow, lamb, calf, buffalo, yak, sheep, goat, camel, alpaca, cervidae, equidae or combinations thereof. At least one dried fruit is selected from peaches, blueberries, raisins, dates, figs, plums, apples, oranges, pineapples, pineapple, tejocote, guava, mango, tamarind or combinations thereof.

The nutritionally acceptable excipient and/or vehicle is selected from water, milk of animal and/or vegetable origin, fruit and/or vegetable juices, fruit and/or vegetable juices or combinations thereof.

### Example 1

This novel integrated nutritional support system comprises the stages of nutrition, metabolic support, support for neurological remodeling, immune stimulation, regulation of inflammation and the oxide reduction mechanism.

Said comprehensive nutritional support system was determined through an exploratory study designed as a randomized, blinded, controlled clinical trial with consecutive case sampling. Patients with cerebral palsy with spastic diparesis and GMFCS III (Gross Motor Function Classification System III) were selected and enrolled in this study (over a 3-year period) and treated at the Centro de Rehabilitación Infantil Teletón (hereafter, CRIT, Teleton Children's Rehabilitation and Inclusion Centers by its acronyms in Spanish) in Tlalnepantla, State of Mexico. The duration of the study was 13 weeks for each participant.

The trial complied with the principles of the Declaration of Helsinki and the Mexican standard NOM-012-SSA3-2012 for scientific research in humans. All procedures were approved by the Research Committee of the Faculty of Health Sciences of the Universidad Anáhuac México Campus Norte.

### Attendees

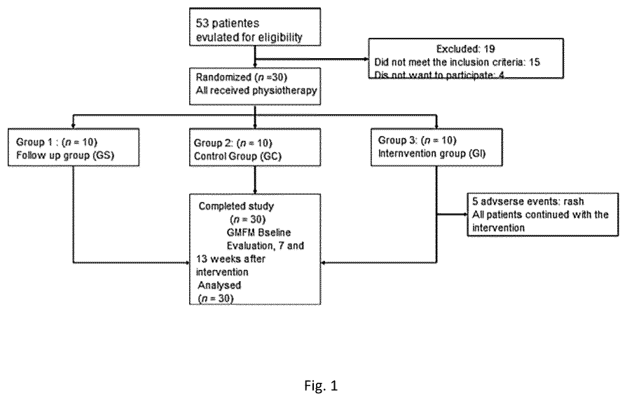

Fifty-three children were interviewed and, of this group, 30 met the inclusion criteria (FIG. 1).

Inclusion criteria: patients with Cerebral Palsy with spastic diparesis and GMFCS III (these patients demonstrated high functionality in the categories of lying/rolling, sitting and crawling), of both sexes aged 4 to 12 years, who had full-time support from a caregiver and who could feed themselves orally.

Non-inclusion criteria: patients with Cerebral Palsy who present another catabolic disease that may increase the risk of malnutrition (renal, cardiovascular, pulmonary, hepatic or immunological disease), who present infections or who received antibiotic treatment at least 15 days before starting the study. Patients who have received therapy with botulinum toxin or muscle relaxants in the last 6 months. Patients with Cerebral Palsy who will present severe gastroesophageal reflux or with any type of surgery performed in the last 9 months and patients who walk alone.

### Group Assignments

Once the 30 patients were selected according to the inclusion criteria, the patients were randomly assigned into three groups: 1) Follow-up group (FG, n=10), only their usual diet was controlled; 2) Control group (CG, n=10 10), they were dewormed (Deworming was applied in the control and intervention groups, it is not part of the nutritional support) and received the nutritional therapy recommended by the WHO; 3) Intervention group (IG, n=10), they were dewormed and received the comprehensive nutritional support system. Because parasitosis is a common variable that could affect nutrient absorption between groups and, as part of our hypothesis relates to the positive effect that deworming could have on supplement absorption, it was decided to contrast the IG 20 group versus a dewormed (CG) and a non-dewormed (FG) group.

All participants received Bobath physiotherapy, which is mainly used at CRIT for these patients, in which the basis of treatment consists of assessing the patient from the prism of neuroscience and neurophysiology and, at the same time, applying knowledge of motor control, motor learning, neuroplasticity, the sensorimotor system and the musculoskeletal system.

motor control, motor learning, neuroplasticity, the sensorimotor system and the musculoskeletal system.

### Procedures

Once the patients with Cerebral Palsy were selected in the company of their parents and/or caregivers and/or guardians, the protocol was explained to them along with the delivery of their informed consent forms. Upon entering the study, the nutritional history and the GMFM scale score were obtained and recorded. GMFM (Gross Motor Function Measurement) scale score of each patient was obtained and recorded. Thereafter, patients with Cerebral Palsy were randomly assigned to the 3 groups (FG, CG and IG). The energy calculation for the CG and IG groups was performed using Krick's formula (50% carbohydrate, 30% lipid and 20% protein).

Parents and/or caregivers and/or guardians of the participants were trained on the project, procedures, as well as feeding and supplementation schedules. At the start of treatment, the CG and IG groups were dewormed with nitazoxanide at a dose of 7.5 mg/kg every 12 hours for 3 days.

### Diet, Smoothies and Supplements

The comprehensive nutritional support system comprises of a diet of the type of nutraceutical composition and/or dietary composition and/or dietary supplement and/or food supplement and/or food supplement and/or therapeutic composition for oral administration with therapeutic doses based on smoothies with functional ingredients, non-limiting, selected from vegetables, fruits, cereals, tubers and proteins of animal and/or vegetable origin, such as fish.

Additionally, the present invention is supplemented with glutamine, arginine, folic acid, nicotinic acid, zinc, selenium, cholecalciferol, ascorbic acid, spirulina, vegetable protein, n-3 PUFAs and probiotics (Saccharomyces boulardii; from 0.1 to 500 mg every 12 hours for 3 days in the basal period and in week 7, to correct malabsorption).

To ensure that patients reached the necessary caloric intake, they were instructed to drink two smoothies in the morning and one in the evening. Parents and/or guardians prepared smoothies at home and consumed them immediately after preparation. A smoothie-based diet was chosen, preferably but not limited to, as it facilitates the absorption of nutrients in the intestine. Sachets comprising the smoothie ingredients were provided to encourage smoothie consumption and facilitate adherence to therapy. GI supplements were mixed in sachets and administered in at least three smoothies: smoothies 1 and 3 comprise extra virgin olive oil and/or grapeseed oil from 0.5 mL to 15.0 mL, amaranth from 0.5 to 2.0 tablespoons, oatmeal from 0.5 to 2.0 tablespoons, avocado from 0.25 to 0.5 of a large-medium piece, banana from 0.5 to 2.0 large-medium pieces, egg whites between 1.0 to 3.0 10 units, cinnamon between 0.5 g to 2.0 g and almond milk between 100.00 mL to 500.00 mL, equivalent to between 500 to 850 kcal, between 10.0 g to 20.0 g of protein, between 5.0 g to 20.0 g of lipids and between 40.0 g to 80.0 g of carbohydrates. Smoothie 2 comprises celery from 0.25 to 15 1.0 piece of a stalk, oranges from 0.5 to 3.0 pieces, pineapple from 25.0 g to 250.0 g, nopal from 0.5 to 2.0 of a medium leaf piece, parsley from 0.5 to 2.0 piece of a sprig, radish from 0.25 to 1.5 medium piece and ginger from 0.5 g to 6.0 g (from 50 kcal to 20 300 kcal, from 0.5 g to 6.0 g of protein, from 0.5 g to 6.0 g of lipids and from 10.0 to 75.0 g of carbohydrates which is drunk in the morning together with the smoothie 1.

The meals-diet during treatment comprised vegetable soup, fish broth, lentil soup, green salad (without dressings), toasted sesame, grilled fish (at least 3 times per week), mushrooms, shiitake mushrooms, egg whites, cereals with legumes, pico de gallo, tortillas and/or steamed brown rice, quinoa and/or amaranth, at least ¼ piece of avocado, natural water from various fruits, and all foods are on demand.

During the therapeutic nutraceutical treatment, two times a day between meals are used, where kefir or natural non-fat yogurt is administered orally to the patient with a dose of at least 250.00 mL, a portion of amaranth of at least two tablespoons and between 5 to 15 almonds. Vegetables and fruits on demand with at least 50.00 grams of purple and red fruits. In order to achieve satiety in the patient.

The considerations for the patient are: no hunger, no fasting, daily physical therapy for at least 60 minutes with the help of a parent and/or guardian in combination with visits to CRIT for physiotherapy, drinking 2 liters of water daily, no hot food or hot liquids, no fasting water, no junk food, no alcohol or tobacco and no irritating foods.

### Follow Up

Participants attended CRIT twice a week for physiotherapy and once a week for a clinical and nutritional check-up. At the latter, patients handed in their food diaries and supplement sachets—to be counted—to assess their adherence during treatment. On days when patients attended physiotherapy, they were monitored to ensure that they ate breakfast, snack and lunch.

### GMFM Evaluation

The GMFM scale was performed at baseline, week 7 and 13 after the intervention. It was administered by CRIT staff. The evaluators were the blinded aspect of the study, as they did not have access to any information about the treatment each child received.

This scale evaluates five general parameters: 1. Lying (decubitus) and turning (GMFAV), 2. Sitting (GMFB), 3. The scoring system consists of 88 items and each item is scored according to the following criteria: 0=No, 1=Onset, 2=Partially complete, 3=Complete, NE=Not assessed.

### Statistical Methods

The Shapiro-Wilk test was used to determine the distribution of the data. Data analysis was performed using the Mann Whitney U test or the Kruskal Wallis test followed by Dunn's post hoc test.

In order to estimate the effect size for the standing and walking assessments, Rosenthals' r, an effect size test for non-normally distributed data, was used. This test can be used together with the results of the Mann Whitney U test. The significance level was considered <0.05 in all cases.

### Size of the Sample

Fifty-three patients were intervened, for this group only 30 patients were recruited and studied. This research is based on an exploratory study and the sample size was determined by the feasibility of recruitment. A sample of 10 children per group was established considering that the number of patients per year in the CRIT with the required criteria for the study fluctuates between 10 and 15 patients.

This sample size allows the detection of an effect size of 0.1 or greater. In which, to reach the established sample, a period of three years is required. Table 1.

TABLE 1
Clinical characterization of patients with cerebral palsy.

| | | Age | GMFD | GMFE |
|---|---|---|---|---|
| | | (years) | Baseline | Baseline |
| Group | Gender | 7.2 1.9 | score | score |
| FG | F | 9.6 | 7.7 | 11.4 |
| FG | F | 4.8 | 7.7 | 14.2 |
| FG | M | 5.7 | 7.7 | 18.3 |
| FG | M | 4.5 | 17.7 | 18.3 |
| FG | M | 6.8 | 5.1 | 18.3 |
| FG | M | 7.5 | 7.7 | 8.8 |
| FG | F | 5.1 | 27.7 | 25.0 |
| FG | F | 9.3 | 7.7 | 11.1 |
| FG | M | 5.5 | 37.7 | 16.7 |
| FG | F | 6.3 | 7.7 | 12.8 |
| CG | M | 9.8 | 23.1 | 16.7 |
| CG | M | 9.2 | 7.7 | 12.9 |
| CG | F | 7.3 | 7.7 | 9.2 |
| CG | M | 9.7 | 15.4 | 25.0 |
| CG | F | 7.5 | 38.5 | 29.2 |
| CG | M | 5.9 | 7.7 | 22.2 |
| CG | M | 8.1 | 7.7 | 16.7 |
| CG | M | 4.6 | 18.0 | 19.4 |
| CG | M | 12 | 7.7 | 13.9 |
| CG | M | 6.1 | 23.1 | 15.3 |
| IG | F | 7 | 1.0 | 8.3 |
| IG | F | 6.4 | 51.1 | 43.1 |
| IG | M | 11 | 7.7 | 8.3 |
| IG | M | 6.2 | 7.7 | 22.2 |
| IG | F | 6.8 | 7.7 | 18.1 |
| IG | M | 8 | 7.7 | 25.0 |
| IG | F | 5.8 | 18 | 22.2 |
| IG | M | 4.8 | 20.5 | 19.4 |
| IG | M | 7.9 | 25.6 | 25.0 |
| IG | F | 7.6 | 20.5 | 11.1 |

FG = follow-up group; CG = control group; IG = intervention group; F = female; M = male; GMFD = gross motor standing function; GMFE = gross motor walking function.

Adherence to the course of treatment was strictly supervised, more than 90% of the patients complied with the intervention therapy. Each patient was asked to keep a food diary, which allowed us to establish their caloric intake per day. This was around between 2000 kcal and 3500 kcal for GC and GI patients and between 750 kcal and 2000 kcal for GS patients.

### Baseline Results

The baseline values of GMFM parameters (lying, sitting, crawling, standing and walking), were not statistically different between the groups studied: lying (FG: 92.75±3.44, GC: 93.34±2.92, GI: 84.13±15 8.87; mean±SEM; p=0.950; Kruskal Wallis followed by Dunn's post hoc test), sitting (FG: 61.34±5.24, CG: 74.15±5.49, GI: 75.16±8.09; p=0.089), crawling (FG: 49.52±5.56, CG: 47.38±8.90, GI: 61.86±7.79; p=0.248), standing (FG: 13.44±3.46, CG: 15.66±3.25, GI: 16.79±4.58; 20 p=0.666), walking (FG: 15.49±1.51, CG: 18.05±1.90, GI: 20.27±3.24; p=0.365).

### Standing and Walking Evaluations

Throughout follow-up, the parameters of lying down, sitting and crawling showed no significant differences between the groups.

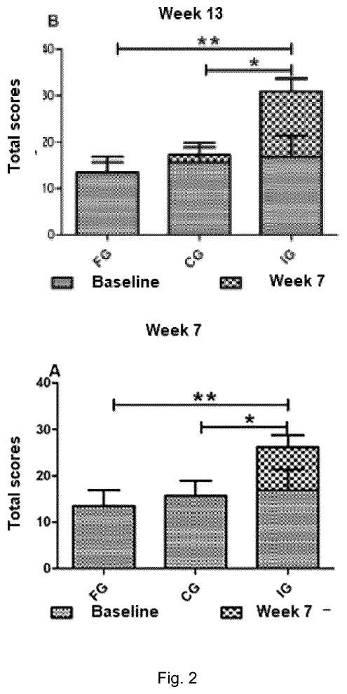

FIG. 2 shows the comparison of patients with FG, CG and IG in the standing parameter at 7 and 13 weeks after the intervention. A total score was considered for these assessments. Seven weeks after the intervention, GI patients had a significant improvement in standing (26.19±8.2; mean±SD) compared to GS (10.1±4.9; p=0.0004, Mann Whitney U-test) and CG (12.98±7.2; p=0.003, Mann Whitney U-test.

To strengthen these results, Rosenthal's r, a statistical assessment that evaluates the effect size (clinical efficacy of the treatment), was calculated. The results showed a large effect size for the standing parameter in the IG group (r=0.64). Thirteen weeks after the intervention, the improvement continued to be statistically different in GI patients (30.90±8.7) compared to GS (13.32±7.4, p=0.0002, Mann Whitney U test) and GC (17.29±8.1, p=20 0.003 Mann Whitney U test). In this case, we also calculated Rosenthal's r and found a large effect in the IG group (r=0.61).

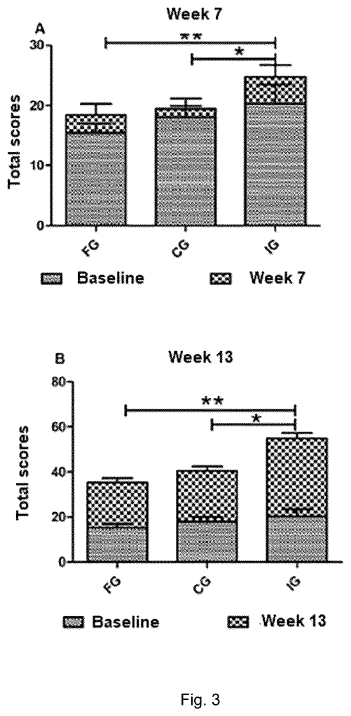

FIG. 3 shows the comparison between GS, GC and GI patients in the gait parameter at 7 and 13 weeks after the intervention. The total score was considered. Seven weeks after intervention, there was a significant difference in gait improvement in GI patients (24.65±6.1) relative to GS (18.47±5.7; p=0.01, Mann Whitney U-test) and GC (19.45±5.6; p=0.03, Mann Whitney U-test).

Rosenthal's r test showed a large effect size in the GI group (r=0.37). After thirteen weeks, GI patients continued to show significant improvement (34.68±7.3) compared to GS (19.86±6; p=0.0003, Mann Whitney U test) and GC (22.51±5.9; p=0.001, Mann Whitney U test) patients. Rosenthal's r was also calculated for this data set. The results showed a large treatment effect size in GI patients on the gait parameter at thirteen weeks (r=0.67). Finally, of this group of patients (GI), five managed to walk alone (without support from anyone else; demonstrated with videos as supplementary material). No patient in the GS or GC groups managed to walk independently.

### Percentage of Evolution

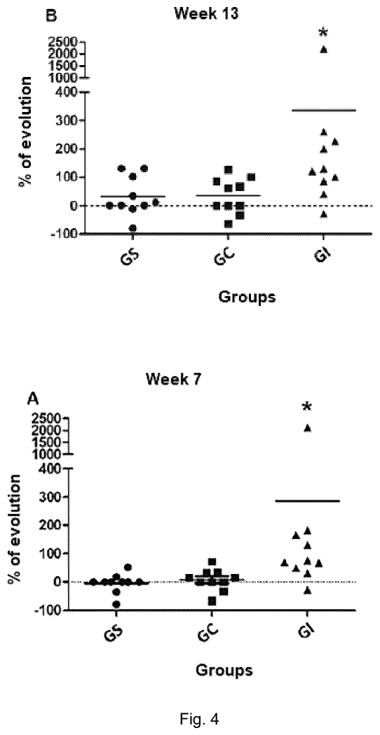

FIG. 4 shows the comparison of the percentage of standing evolution between the groups studied. The percentage was calculated according to the formula: corresponding final value (7 or 13 weeks)÷initial value−1×100).

Therefore, we are reporting the percentage of evolution after 7 or 13 weeks of intervention. GI patients showed a significant improvement in percent evolution compared to GS and CG patients (p=0.025, Kruskall Wallis followed by Dunn's post hoc test). Evolution to the thirteenth week also revealed a significant difference in improvement in GI patients compared to GS and GC patients (p=0.03, Kruskall Wallis followed by Dunn's post hoc test).

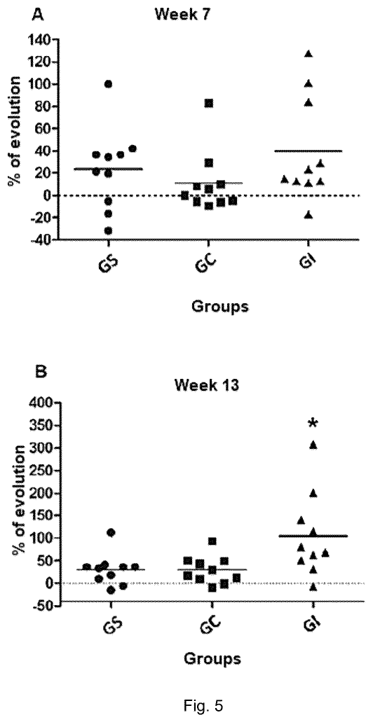

When the walking parameter was analyzed, the evolution from baseline to the seventh week did not show any significant advantage for any of the groups (FIG. 5; p=0.15, Kruskall Wallis followed by Dunn's test). However, in the evolution observed up to the thirteenth week, patients with GI resented a significant improvement, compared to patients with GS and GC (p=0.03, Kruskall Wallis followed by Dunn's test).

With the above analysis, the effect of the present comprehensive nutritional support system in improving motor function in patients with cerebral palsy was evaluated. The findings showed that the integrated nutritional support system promoted a significant improvement in the standing and walking parameters of the GMFM scale. In addition, the therapy with the integral nutritional support system made half of the patients studied walk on their own. This is a relevant finding since the improvement in motor function was, in a shorter time, superior to that observed in patients with Cerebral Palsy submitted only to conventional therapy.

From baseline assessment (7 weeks after initiation of therapy), the performance of the GI patients was better than that observed in the GC and GS groups. Improvement in the treated GI patients was evident thirteen weeks after initiation of therapy. The results observed in the treated GI patients were beyond those reported prior to using physical therapy alone (2-3% annual improvement).

The result obtained is due to the administration of various nutrients and supplements that are necessary to compensate for the metabolic, nutritional and neurological deficiencies that patients with Cerebral Palsy present.

The improvements observed in standing and walking were related to a reduction in spasticity. Changes in hand movements were also observed in the majority of GI patients, resulting in the release of the thumb and the “claw hand” gripper. Spasticity is related to a lesion in the Pyramidal System (PS), so any improvement in these areas is associated with a remodeling of the PS and thus, the Central Nervous System, particularly in the motor and premotor cortex areas, as well as in the brainstem.

This specific remodeling is related to myelination, neurogenesis, promotion of neurotransmitter release or even ganglion generation. The PS consists of approximately one million fibers, mainly myelinated, whose origin is in the primary motor and premotor cortex (80% of the fibers). Its function is to control voluntary movements, both fine and gross motor, so that any alteration of these fibers' causes, among other alterations, spasticity.

Therefore, any therapy based on PS remodeling is contributing to decrease spasticity and, therefore, to improve fine and gross motor skills in patients with Cerebral Palsy.

The induction or increase of plasticity phenomena or even regeneration of neurons by providing substrates such as nutrients is highly feasible. Several different nutrients, such as n-3 PUFA (EPA and DHA), have been used to stimulate plasticity and new neuronal cell bodies, and nutrients such as zinc, ascorbic acid, spirulina, arginine, n-3 PUFA and vitamin D have been used in neuronal cell body repair and neuronal regeneration.

On the other hand, the production of neurotransmitters such as serotonin and GABA can be increased by using probiotics, nicotinic acid, cobalamin, pyridoxine, folates, zinc, ascorbic acid and tryptophan. Thus, regulation of serotonin and GABA production may help control spasticity and thus fine and gross motor functions. In addition, the use of probiotics has become relevant to enhance gut signaling and promote neurotransmission, metabolism, immunity and inflammation by stimulating the gut-brain microbiota axis.

Currently, studies on the gut microbiota have become relevant in the treatment of various neurological disorders, including autism and alterations in memory and neuronal regeneration.

Also, the simultaneous supplementation of probiotics and prebiotics, such as inulin and other nutrients such as glutamine, induces the generation of short-chain fatty acids that modulate inflammation and favor the regeneration of the nervous system, as well as the restoration of enterocytes and muscle synthesis.

Finally, it has been demonstrated in studies in mice that glutamine together with probiotics inhibits nitric oxide, reducing the levels of proinflammatory factors such as TNF alpha, IL6, IL8 and reactive oxygen species (ROS). Such evidence shows that combining probiotics with other nutrients to create symbiosis induces positive effects on immunity, restoring the mucosa and the nervous system.

### Example 2

The present novel comprehensive nutritional support system comprises the stages of nutrition, metabolic support, immune system support, regulation of inflammation and oxidation-reduction potential.

Said nutritional support system is focused on strengthening the immune system and reducing mortality due to COVID-19 disease caused by the SARS-CoV-2 virus, which has been classified as mild, severe, non-pneumonic and asymptomatic, depending on the clinical manifestations and radiological evidence of the pneumonic process. The presence of comorbidities such as obesity, cardiovascular disease, high blood pressure, type 2 diabetes mellitus and immune deficiency states have been associated with more severe disease.

In Mexico, according to the interim algorithms for the care of COVID-19 are classified according to the time of evolution, signs and symptoms, the application of different clinical scales and laboratory findings in: stage I (early infection) outpatient management, stage II (pulmonary phase) medical review and evaluation of hospitalization; and stage III (hyperinflammatory phase, cytokine storm) the patient requires hospitalization in reconversion areas.

Transmission of the SARS-CoV-2 virus occurs through the air, or through fomites, airborne spread is due to respiratory droplets reaching 1-2 m generated by talking, coughing and sneezing, the virus can remain viable for 24-72 hours on surfaces which allows transmission by contact with such fomites and touching eyes, nose or mouth; SARS-CoV-2 enters human cells through the respiratory tract by binding to angiotensin-converting enzyme 2 (ACE2), expressed in alveolar cells, cardiac myocytes, vascular endothelium and other cells. Infection occurs in 3 steps: binding to the receptor, conformational changes in glycoprotein S, and proteolysis of cathepsin L within endosomes. Upon entering the host cell, the virus releases the RNA genome into the cytoplasm, subsequently attaches to ribosomes and initiates viral genome translation.

SARS-CoV-2 can affect different cellular levels.

Clinical manifestations of COVID-19 are nonspecific, may remain asymptomatic, mild clinical manifestations such as fever, cough, sore throat, fatigue, dyspnea, myalgia, headache, vomiting and diarrhea. The presentation and severity usually vary depending on some factors such as: age older than 65 years, chronic comorbidities and compromised immune system. In these cases, the patient may be at increased risk of developing organ dysfunction, including shock, acute respiratory distress syndrome (ARDS), acute cardiac injury and acute kidney injury, resulting in a higher mortality rate (15). Symptoms of COVID-19 infection appear after an incubation period of 5-6 days, the time period between the onset of symptoms and death ranges from 6 to 41 days, with a median of 14 days.

Damage by immune mechanisms is caused by a storm of cytokines, mainly inflammatory, that activate T lymphocytes, macrophages and endothelial cells, with consequent vascular permeability, activation of the complement cascade and coagulation. In patients with severe COVID-19, there is a decrease in the absolute number of circulating CD4+ cells, CD8+ cells, B cells and NK cells; a decrease in monocytes, eosinophils and basophils has also been reported. SARS-Cov-2 requires in a keyway the association with S-adenosyl-L-methionine (SAM), with its nsp 14N7-Mtase regions. When SAM is transformed into S15 adenosylhomocysteine (SAH) and this is in contact with SAH hydrolase (SAHH), homocysteine is obtained, which is an intermediate that stimulates oxidative stress and is recycled by re-methylation and trans-sulphuration in humans.

In a study published in the journal Nature Communications, the focus was on understanding the homocysteine-related regulatory mechanisms that stimulate the angiotensin II receptor type II. Thus, SARS-CoV-2 utilizes the increased activation of ACE2.

Regarding nutritional status, lymphopenia, which is a marker of malnutrition, is a poor prognostic factor in patients with COVID-19. Circulating albumin levels should not be considered as a nutritional marker in patients with active inflammatory response, as it was recently reported that low levels of prealbumin predict progression of ARDS. The acute phase response, induced by inflammation and infections, alters lipid metabolism, decreasing serum HDL and increasing triglycerides, which initially may help fight infection, but increases the risk of atherosclerosis. Reducing inflammation has been shown to result in the return of the lipid profile to normal. It has been shown that, in patients with muscle wasting due to a medical condition, the use of glutamine, arginine and HMB (beta hydroxy beta methyl butyric acid) can decrease muscle loss because they can promote the production and decrease in protein degradation. Since arginine improves cell size and protein synthesis and glutamine activates cell division.

Treatment for patients with COVID-19 is generally supportive and symptomatic, depending on the severity of symptoms, although there is no specific treatment so far, most patients require supplemental oxygen. Pharmacological treatment varies depending on the patient's complications and symptoms, including drugs such as ceftriaxone, azithromycin, oseltamivir, ivermectin, nitazoxanide, ritonavir, lopinavir, remdesivir, favipiravir, among others; together with the nutritional therapy recommended by the WHO, calculating the energy requirement per weight at 27-30 kcal/kg/day; protein requirement up to 1.5 g/kg/day in case of not having chronic renal insufficiency; carbohydrate and lipid requirement with a range of 30:70 in patients without respiratory insufficiency and 50:50 in patients with respiratory insufficiency.

Nutritional status influences the evolution of patients with COVID-19, but so far there is no information on the impact of early nutritional support in patients with COVID-19 before ICU. It is known that hospitalized patients present a significant degree of inflammation at the systemic level, which causes inappetence causing an inadequate nutritional status and this could lead to respiratory failure requiring ventilatory support, therefore, proper nutrition supports the optimal functioning of the immune system.

### Evidence of Specific Nutrients

Probiotics are beneficial in the prevention of acute upper respiratory tract infection, helping to counteract dysbiosis in patients with COVID-19. Specifically, Saccharomyces boulardii, also increases IgA levels in the intestine reducing the pro-inflammatory response. On the other hand, the beta-glucans of the cell wall of brewer's yeast (Saccharomyces cerevisiae) stimulate the immune system, especially that related to inflammatory responses (leukocytes and epithelial cells) and the reticuloendothelial system. Nutrition is one of the most important axes in the therapy of different diseases, due to its selective action on one or several functions of the organism, improving the course of some diseases, including viral diseases. This premise supports the hypothesis that certain nutrients favor the immune response with respect to the common features currently presented in patients with COVID-19. The SARS-CoV-2 viral genome encodes for the nsp12 protein, which harbors RNA-dependent RNA polymerase (RdRP) activity. It has been shown that vitamin B12 (methyl cobalamin) can bind to the active site of the nsp12 protein, preventing association with RNA and thus inhibiting the RdRP activity of nsp 12. On the other hand, administration of 2-500 UM of Zinc significantly inhibits influenza replication in vitro, through inhibition of RNA-dependent RNA polymerase (RdRp), with a similar effect occurring with SARS-CoV, where binding and elongation of the RdRp template is inhibited (47). In addition, C-phycocyanin, an extract found in Spirulina maxima, acts by blocking the hemagglutination of virus particles to inhibit influenza virus strains, thus helping to prevent morbidity in airway diseases. Protein D1, derived from Omega 3, reduces virus replication of some strains of influenza viruses, so omega 3 is also considered an important nutrient when considering a dietary regimen with antiviral action. Arginine, resveratrol is able to decrease apoptosis induced by MERS-COV and inhibit infection caused by it, prolonging cell survival after infection. These antiviral mechanisms are due to the fact that resveratrol activates the ERK 1/2 signaling pathway and promotes cell proliferation by enhancing SIR1 signaling, both of which are related to cell survival and DNA repair in response to DNA damage. The release of proinflammatory cytokines such as TNFα, IL-6, IL-8 and IFN (interferon Alpha, Beta and Gamma). It correlates with the severity of the disease. The administration of vitamin D especially in its active form 1,25, dihydroxycholecalciferol [1,25 (OH) 2D3], causes a modulation in the immune system and antiviral activity, by reducing the expression of proinflammatory cytokines and increasing the expression of anti-inflammatory cytokines such as TNFα, IL-6, IL-8 and IFN, by macrophages through regulation by vitamin C. In a study by Khare et al, they observed that treatment of human lung epithelial cells with 100 or 30 nM of 1α, 25 (OH) 2D before or after H1N1 exposure significantly decreased TNFα, IFNβ and IFNγ levels and IL-8 and IL-6 levels. Vitamin D deficiency (plasma 25 (OH) D levels <50 nmol/L) has been identified in patients with acute respiratory distress syndrome (ARDS).

25-hydroxycholecalciferol is a stable marker of vitamin D status that can be measured in blood. This 20 vitamin immunomodulates the innate and adaptive immune system, which is useful against the cytokine storm caused by SARS-Cov-2, since its active metabolites modify NF-κB signaling in macrophages, reduces the synthesis of proinflammatory mediators (TNFα, IL-6 and MCP-1) and decreases monocyte and macrophage recruitment, lowering overall tissue inflammation, in addition, it has been documented to have an antifibrotic effect in lung tissue. Omega 3, derived from the formation of docosahexaenoic acid (DHA), reduces extremely high inflammatory parameters, as it regulates lymphocyte proliferation, cytokine production and neutrophil chemotaxis. Folic acid mediates the production of proinflammatory cytokines and the Th-1 cytokine-mediated immune response. Therefore, its deficiency decreases the antibody response to various antigens, including the blastogenic response of T lymphocytes to certain mitogens, and the capacity of CD8 T lymphocyte cells.

There is evidence that magnesium plays a key role in the immune response as a cofactor for immunoglobulin synthesis, as well as a relationship between magnesium deficiency and increases in proinflammatory cytokines. Intracellular free glutamine in skeletal muscle is known to be markedly reduced in the stress response to surgery, trauma and inflammatory states; thus, reduced plasma glutamine values have been detected in hospitalized patients with emphysema, COPD and evidence of muscle wasting. SARS-CoV-2 enters the cell through its surface proteins; however, this protein has to be divided into 2 domains, where furin intervenes. It has been determined that folic acid inhibits furin activity, preventing the virus from entering the cell.

COVID-19 is a disease that spreads rapidly, with a high mortality rate, which has caused the collapse of health systems and the concern of their personnel; for this reason it is of vital importance to implement a comprehensive treatment to avoid complications of the disease, reduce the length of hospital stay and provide a rapid recovery. Treatment costs can vary dramatically, from $500.00 Mexican pesos per admission to $1,512,000.00 Mexican pesos, considering an average hospital stay of 12 days, with costs substantially higher than other infectious diseases. It has been reported that approximately 26.4% of hospitalized patients recovered and were discharged from the hospital, and 46.4% improved and were reclassified. Among the severe cases reported, 13.4% have died. Various lines of treatment have been addressed, but feeding is only considered as part of the caloric intake of patients and not as part of the treatment. This study evaluated the nutritional status of patients with COVID-19 when starting a nutritional therapy based on supplements such as Omega 3, Zinc, Selenium, Vitamin D, Glutamine, Spirulina, prebiotics among others, which have been shown to improve the immune response, especially against viral infections and thus, demonstrate the reduction of complications in patients with adequate nutritional support, reduce the days of hospital stay, disease progression, percentage of progression to mechanical ventilation and mortality. Being the results transcendental for its application, even worldwide, this study provides scientific evidence to establish new strategies to improve the clinical evolution of patients with COVID-19 and the mortality rate. Not only the patient and his family will benefit, but also the health system, by avoiding its collapse. This study is feasible given that it has the material and human resources to be conducted and is pertinent given that it seeks to contribute to the study of new nutritional therapies to reduce complications and mortality in patients with COVID-19.

The present nutritional support system reduces complications and mortality in COVID-19 positive patients with comorbidities (type 2 DM, HAS, overweight/obesity with BMI <35) in stage III, with a better benefit than that achieved with conventional nutritional treatment.

The correlation of biochemical and inflammatory factors with the evolution of the study groups, body composition with the clinical evolution of the patients, days of hospital stay, disease progression, percentage of progression to ventilator and mortality were established as predictive indexes of patient improvement.

### Methodology

Type of study and overall design: Blinded, randomized controlled clinical trial.

### Universe of Study

Patients diagnosed with COVID-19 by PCR test, who are attended at Centro Médico ISSEMYM (Instituto de Seguridad Social del Estado de México y Municipios (Social Security Institute of the State of Mexico and Municipalities, by its acronyms in Spanish) Toluca Arturo Montiel Rojas, located at Paseo Tollocan, Av. Baja Velocidad km 575, Barrio de Santa Clara, Toluca de Lerdo, Mexico; both sexes, with presence of comorbidities (DM typom2, HAS, overweight or obesity BMI <35) and in stage III of the disease.

Selection and sample size: The sample size was set at 37 participants per group. Three additional patients are added for possible dropouts, leaving an n of 80 patients. The participants will be divided into 2 groups of 40 participants each.

### Inclusion and Exclusion Criteria

### Inclusion Criteria

1. Patients admitted to the Centro Médico ISSEMYM Toluca Arturo Montiel Rojas, diagnosed with COVID-19 confirmed by PCR.
2. Patients in need of supplemental 02 with nasal prongs or mask—reservoir for 02 saturation <90% and respiratory distress.
3. With concomitant diseases such as cardiovascular disease, diabetes mellitus 2, high blood pressure, overweight or obesity BMI <35.
4. Both sexes
5. Over 30 years old.
6. Tolerates oral feeding
7. Signing the informed consent letter.

### Non-Inclusion Criteria

1. Who requires mechanical ventilatory assistance.
2. Have neurological or neurodevelopmental disorders
3. Severe gastroesophageal reflux and/or dysphagia.
4. Patients with any type of surgery performed less than 3 months ago.
5. Allergies to any ingredient in the study treatment.
6. Who presents acute malnutrition.
7. Who requires admission to the Intensive Care Unit.
8. Who has criteria for sepsis or septic shock.
9. Respiratory rate >30/min
10. Glycemia greater than 240 mg/dl

### Elimination Criteria:

1. Detachment to treatment.
2. Admission to the ICU for any reason.
3. Patients who do not tolerate the oral route.
4. Reactions to treatment that compromise the health of patients.
5. Glomerular filtration rate less than 60 ml/min/1.73 m2
Sampling: Consecutive cases.
Assignment: Systematic randomized assignment by means of a sequence of random numbers constructed with the Excel program divided into two groups. Once this is obtained, each patient will be assigned to a group as they enter, thus integrating the control group and the intervention group.

### Procedure

Once the participants had been selected, the patients and/or family members were spoken to in order to explain the protocol and obtain the signatures of the informed consent letters.

Once the patients had been admitted to the study, the following was performed:
1. Diagnostic test for COVID-19 by PCR.
2. Complete medical and dietary history
3. Complete blood count: leukocytes, neutrophils, eosinophils, basophils, lymphocytes, monocytes, erythrocytes, red blood cell count, hemoglobin, hematocrit, mean corpuscular volume (MCV), mean hemoglobin concentration (MHC), mean corpuscular hemoglobin concentration (MCHC), platelets and mean platelet volume (MPV).
4. Blood chemistry (glucose, urea, urea nitrogen (BUN), creatinine, BUN/creatinine ratio, uric acid, calcium, phosphorus, sodium, potassium, chloride, magnesium, glomerular filtration rate (GFR), triglycerides, total cholesterol, low density lipoproteins (LDL), high density lipoprotein (HDL), very low density lipoprotein (VLDL), total lipids, total, direct and indirect bilirubin, alanine aminotransferase (ALT), aspartate aminotransferase (AST), gamma glutamyl transpeptidase (GGT), lactate dehydrogenase (LDH), total proteins and albumin.
5. D-dimer.
6. Coagulation times, bleeding time and prothrombin time
7. Fibrinogen.
8. Serum electrolytes: sodium, potassium, magnesium and chlorine.
9. Serum 25 hydroxycholecalciferol levels.
10. Measurement of % muscle mass, % fat, BMI, weight, height.
11. Assignment to the research group on a random basis, which will have the following features;
a) CO: Basal Energy Expenditure (BEE) plus stress factor was calculated using the Harris Benedict equation. The distribution of macronutrients was 50% for carbohydrates, 30% for lipids and 20% for proteins. The food was established according to that established in the nutrition department of the Centro Médico ISSEMYM Toluca Arturo Montiel Rojas.
b) GI: Basal Energy Expenditure (BEE) plus stress factor was calculated using the Harris Benedict equation. The distribution of macronutrients was 50% for carbohydrates, 30% for lipids and 20% for proteins. The food will be established according to what is established in the nutrition department of the Centro Medico ISSEMYM Toluca Arturo Montiel Rojas, together with the following supplementation:
1. B complex vitamins, IM.
2. One sachet of the integral nutritional support system, at least one sachet in the morning and at least one sachet in the afternoon mixed with 400 ml of water each.
3. Between 50 mg and 1000 mg of Saccharomyces boulardii were administered, given with 1 capsule of 50, 100, 250, 400, 500 or 1000 mg every 2, 4, 6, 8, 10 or 12 hours for at least the first 6 days.

The energy balance of the supplements is considered within the designated caloric balance for this group. Follow-up was performed daily for 21 days or earlier, if they are discharged from the hospital due to improvement in evolution, at which time the patient concludes the study and will be followed for a period of 40 days to monitor survival. The evaluators and the treating physicians are blinded to avoid bias (laboratory personnel, nutrition department, medical personnel and nursing personnel). The following items were monitored:
1. Application and consumption of supplementation of the comprehensive nutritional support system as appropriate.
2. Recording of all information in the files.
3. Food diaries of each patient.
4. Lab studies: QS every other day, BH every 3rd day, coagulation times every 3rd day, D-dimer and fibrinogen every 3rd day, serum electrolytes every other day.
5. SpO2%, heart rate, respiratory rate, respiration rate
6. Clinical evaluation and recording of bowel movements.
7. Measurement of % muscle mass, % fat, BMI, weight, height every two days.
8. Average number of days of hospital stay.
9. Intubation and mortality analysis.

### Ethical Considerations

The present clinical trial complied with the guidelines stipulated in the regulations of the General Health Law on Research, article 17 of which considers it to be a minimal risk research study due to the method of assignment and intervention. Considering the level of risk, and in accordance with the provisions of articles 36 and 37 of this Law, the consent was in writing and digitalized by means of a letter of informed consent in which the patient is informed of the objective of the study, the method to be used, the expected benefits and the possible discomforts. The study did not affect the living, moral or physical conditions of the study subjects. The basic principles of the Declaration of Helsinki, the Nuremberg Code and NOM-012-SSA3-2012 on scientific research on humans were not violated, and the international Ethical Guidelines for biomedical research involving human subjects were also followed.

### Results

The baseline GC and GI results showed no significant difference in all the variables analyzed. Kaplan-Meier survival curves were applied to evaluate overall mortality.

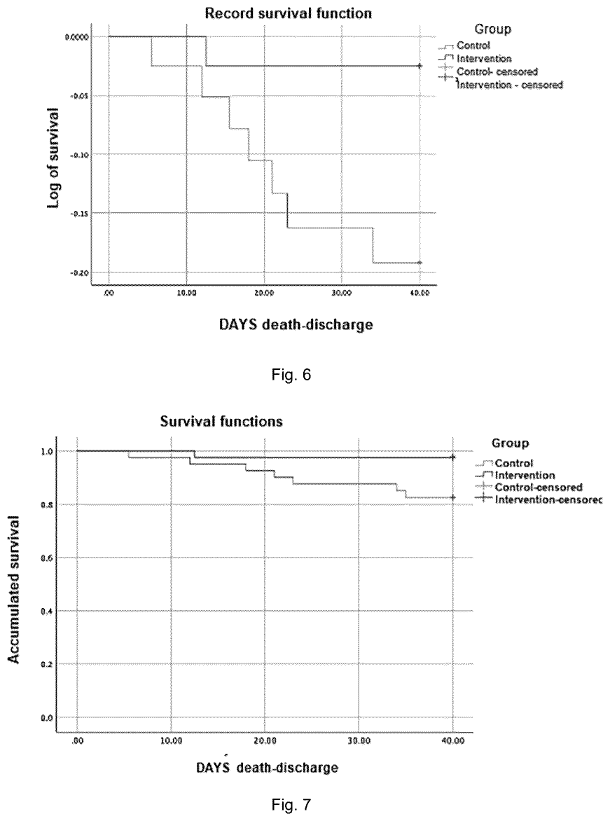

FIG. 6 and FIG. 7 show the analysis of the 80 patients who entered a 40-day observation period, where the survival rate for the CG is 82.5%, with seven of the 40 patients included in this group dying, compared to the IG with 97.5% survival, with one of the 40 patients included in this group dying, with a p value=0.027 obtained by the Log Rank test (Tables 2, 3 and 4).

TABLE 2
Kaplan-Meier case processing for Log Survival
Function and Survival Functions.

| | | |
|---|---|---|
| Total | No. of | Censored |

| Group | N | events | N | Percentage |
|---|---|---|---|---|
| Control | 40 | 7 | 33 | 82.5 |
| Intervention | 40 | 1 | 39 | 97.5 |
| Global | 80 | 8 | 72 | 90.00 |

TABLE 3
Means and medians for survival time. Estimation is
limited to the longest survival time, if censored.

| | 95% confidence interval | | | | 95% confidence interval | | | |
|---|---|---|---|---|---|---|---|---|
| | | Error | Lower | Top | | Error | Lower | Top |
| Group | Estimate | dev. | limit | limit | Estimate | dev. | Limit | limit |
| Control | 36.225 | 1.409 | 33.464 | 38.986 | — | — | — | — |
| Intervention | 39.313 | 0.679 | 37.982 | 40.643 | — | — | — | — |
| Global | 37.769 | 0.801 | 36.200 | 39.338 | — | — | — | — |

TABLE 4
Overall comparisons. Test of equality of survival
distributions for the different Group levels.

| | Chi-square | gl | Foll. |
|---|---|---|---|
| Log Rank | 4.874 | 1 | 0.027 |
| (Mantel-Cox) | | | |
| Breslow | 4.801 | 1 | 0.028 |
| (Generalized | | | |
| Wilcoxon) | | | |
| Tarone-Ware | 4.838 | 1 | 0.028 |

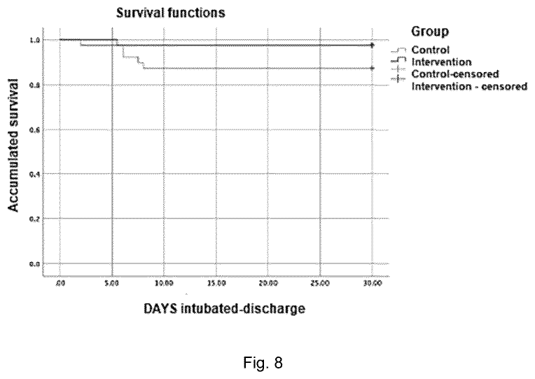

FIG. 8 shows the analysis of the 10 patients who were intubated, where the survival rate for the CG is 29%, with five of the seven patients intubated in this group dying, compared to the IG with 67% survival, with one of the three patients included in this group dying, with a p value=0.08 obtained by the Log Rank test (Tables 5, 6 and 7).

TABLE 5
Kaplan-Meier case processing for Survival
Functions with intubated-high.

| | Chi-square | gl | Foll. |
|---|---|---|---|
| Log Rank | 4.874 | 1 | 0.027 |
| (Mantel-Cox) | | | |
| Breslow | 4.801 | 1 | 0.028 |
| (Generalized | | | |
| Wilcoxon) | | | |
| Tarone-Ware | 4.838 | 1 | 0.028 |

TABLE 6
Means and medians for intubated-high survival time. Estimation
is limited to the longest survival time, if censored.

| | Media | | | | Mediana | | | |
|---|---|---|---|---|---|---|---|---|
| | 95% confidence interval | | | | 95% confidence interval | | | |
| | | Error | Lower | Top | | Error | Lower | Top |
| Group | Estimate | dev. | limit | limit | Estimate | dev | limit | limit |
| Control | 26.910 | 1.296 | 24.370 | 29.450 | — | — | — | — |
| Intervention | 29.388 | 0.605 | 28.202 | 30.573 | — | — | — | — |
| Global | 28.165 | 0.723 | 26.748 | 29.581 | — | — | — | — |

TABLE 7
Overall intubated-high comparisons. Test of equality of
survival distributions for the different Group levels.

| | Chi-square | gl | Foll. |
|---|---|---|---|
| Log Rank | 2.897 | 1 | 0.089 |
| (Mantel-Cox) | | | |
| Breslow | 2.845 | 1 | 0.092 |
| (Generalized | | | |
| Wilcoxon) | | | |
| Tarone-Ware | 2.872 | 1 | 0.090 |

Therefore, it is concluded that the administration of the integral nutritional support system reduces the risk of intubation and mortality of patients with pneumonia and COVID-19 in phase III.

In that situation, the features comprising the present invention are the following:
a. Prevention, treatment and recovery and/or control, such as but not limited to gastrointestinal diseases such as diarrhea, ulcerative colitis, constipation, gastroenteritis or gastric atrophy; restoration of the bacterial flora such as good bacteria of the large and small intestine region; cardiovascular diseases, cholesterol; mental and visual development; selected motor development of cerebral palsy; stimulation and strengthening of the immune system against diseases caused by bacteria, viruses, fungi and other pathogenic microorganisms. Said comprehensive nutritional support system is focused, in a non-limiting manner, on gastrointestinal diseases caused by parasites, mental motor diseases such as cerebral palsy and immune diseases by selected viruses of the group consisting of Arenaviridae, Coronaviridae, Filoviridae, Flaviviridae and Paramyxoviridae, more specifically by selected Coronaviridae group virus of SARS (SARS10 CoV-1 and SARS-CoV-2), MERS, 229E, NL63, OC43, and HKU1.
b. The comprehensive nutritional support system promoted a significant improvement in the standing and gait parameters of the GMFM scale.
c. The therapy with the integral nutritional support system made half of the patients studied walk on their own, demonstrating an improvement in motor function in a shorter time, superior to that observed in patients with Cerebral Palsy submitted only to conventional therapy.
d. Neurological stimulation with improvements in gross motor function observed in standing and walking, related to a reduction in spasticity.
e. The comprehensive nutritional support system shows a striking decrease in mortality, with a significant reduction in the frequency of intubation and in the mortality of intubated patients.
f. The use of the combination as the one intended to be protected provides a nutritional effect that is indicated as a coadjuvant treatment of chronic degenerative diseases, infections and allergic conditions.
g. The participation of PUFA, zinc, ascorbic acid, spirulina or arginine is essential to repair neurological function.
h. Administration of the comprehensive nutritional support system significantly reduces the risk of intubation and significantly reduces mortality in intubated patients with pneumonia and COVID-19.

1. A comprehensive nutritional support system suitable for a human being comprising nutrition, metabolic support and neurological remodeling support stages characterized in that it comprises the administration of a nutraceutical composition and/or dietary composition and/or food supplement and/or food complement and/or therapeutic composition at therapeutic doses in the form of powder and/or lyophilized powder and/or semi-solid and/or liquid and/or semi-liquid and/or semi-liquid and/or in a natural state, to be administered orally, of: at least one vegetable, at least one fruit, at least one cereal, at least one tuber and at least one protein of animal and/or vegetable origin, at least one lipid of animal and/or vegetable origin, at least one amino acid, at least one carbohydrate, at least one vitamin, at least one mineral and/or trace element and/or micronutrient, at least one cyanobacterium, at least one protein of animal and/or plant origin, at least one n-3 PUFAs, at least one probiotic and/or at least one prebiotic, at least one seed, at least one cereal, at least one tree and/or tree bark and/or tree stem and/or tree leaves and/or tree roots, at least one plant and/or plant stem and/or plant leaves and/or plant roots, at least one vegetable and/or animal milk, at least one animal and/or vegetable lipid, at least one polyunsaturated fatty acid, at least one cereal and/or cereal derivative and/or at least one nut and/or at least one nutritionally acceptable drug and/or excipients for the treatment of gastrointestinal diseases, mental motor diseases such as cerebral palsy and virus immune diseases selected from the group consisting of Arenaviridae, Coronaviridae, Filoviridae, Flaviviridae and Paramyxoviridae, more specifically by viruses of the Coronaviridae group selected from SARS (SARS-CoV-1 and SARS-CoV-2), MERS, 229E, NL63, OC43, and HKU1.

2. A comprehensive nutritional support system suitable for a human being according to claim 1 characterized in that it is administered in a semi-liquid oral form of at least two smoothies per day, wherein smoothie A. comprises extra virgin olive oil and/or grape seed oil of between 0.5 mL to 15.0 mL, amaranth 0.5 to 2.0 tablespoons, oatmeal 0.5 to 2.0 tablespoons, avocado 0.25 to 0.5 of a medium-large piece, banana 0.5 to 2.0 medium-large pieces, egg whites 1.0 to 3.0 units, cinnamon 0.5 g to 2.0 g and almond milk 100 mL to 500 mL, equivalent to 500 to 850 kcal, 10.0 g to 20.0 g of protein, between 5.0 g to 20.0 g of lipids and between 40 g to 80 g of carbohydrates; smoothie B comprises celery between 0.25 10 to 1.0 piece of a stalk, oranges between 0.5 to 3.0 pieces, pineapple between 25.0 g to 250.0 g, nopal between 0.5 to 2.0 of a medium leaf piece, parsley from 0.5 to 2.0 of a sprig piece, radish from 0.25 to 1.5 of a medium piece and ginger from 0.5 g to 6.0 g 15 (from 50 kcal to 300 kcal, from 0.5 g to 6.0 g protein, from 0.5 g to 6.0 g of lipids and between 10.0 to 75 g of carbohydrates, where smoothie A is administered at breakfast alone or in combination with smoothie B, and smoothie A is administered at dinner.

3. A comprehensive nutritional support system suitable for a human being, in accordance with claim 1, characterized in that it further comprises Saccharomyces cerevisiae, vitamin B12 (methyl cobalamin), zinc, c-phycocyanin from Spirulina maxima, protectin D1 derived from omega 3, arginine, resveratrol, vitamin D active form, vitamin C, omega 3, folic acid, nicotinic acid, magnesium, glutamine, arginine, brewer's yeast and vegetable proteins.

4. A comprehensive nutritional support system suitable for a human being, according to claim 1, characterized in that the delivery system comprises Spirulina maxima, ascorbic acid, folic acid, nicotinic acid and glutamine, n-3 PUFA, vegetable protein, magnesium, zinc, selenium and cholecalciferol, arginine, inulin, resveratrol, and Saccharomyces boulardii.

5. A comprehensive nutritional support system suitable for a human being, in accordance with claim 1, because the condition to be treated is cerebral palsy and COVID-19.
